(19) United States
(12) Patent Application Publication
(10) Pub. No.: US 2017/0000761 A1
(43) Pub. Date: Jan. 5, 2017

(54) PROTECTING TISSUE AND MITIGATING INJURY FROM CHEMICAL EXPOSURE

(71) Applicant: HUMANETICS CORPORATION, Edina, MN (US)

(72) Inventors: Michael D. Kaytor, Maplewood, MN (US); John C. Dykstra, Minneapolis, MN (US)

(21) Appl. No.: 15/039,496

(22) PCT Filed: Nov. 26, 2014
(86) PCT No.: PCT/US14/67563

§ 371 Date: May 26, 2016

Related U.S. Application Data
(60) Provisional application No. 61/909,409, filed on Nov. 27, 2013.
(60) Provisional application No. 61/909,410, filed on Nov. 27, 2013.

(51) Int. Cl. A61K 31/352; A61K 9/00; A61K 9/12

(52) U.S. Cl. CPC A61K 31/352; A61K 9/12; A61K 9/0078; A61K 9/0019

(57) ABSTRACT

Materials and methods for reducing, preventing, or mitigating the effects of exposure to toxic chemical agents.

### CROSS-REFERENCE TO RELATED APPLICATIONS

This application claims benefit of priority from U.S. Provisional Application Ser. Nos. 61/909,409 and 61/909,410, which were filed on Nov. 27, 2013, and are incorporated herein by reference in their entirety.

### TECHNICAL FIELD

This document relates to materials and methods for reducing, preventing, or mitigating the acute and chronic biological effects of exposure to toxic chemical agents (e.g., phosgene, phosphine gas, or sulfur mustard gas).

### BACKGROUND

Exposure to chemical warfare agents or toxic industrial chemicals can have devastating effects on the respiratory system and, without prompt intervention, can result in death. These chemical threats can create hazards to people and the environment both accidently and through terrorist attack. The respiratory tract is one of the primary targets of chemical agent toxicity following inhalation exposure. The inhalation of high concentrations of these agents typically is characterized by life-threatening pulmonary edema, and the long-term effects of chemical agent inhalation can include chronic obstructive pulmonary disease, asthma, fibrosis, and death. Respiratory damage resulting from exposure is dose-dependent and both immediate and latent in onset. There is no antidote or effective treatment that exists for exposure to these chemical agents, and patients are treated by giving supportive medical care to minimize the effects of exposure.

### SUMMARY

This document is based on part on the discovery that compositions containing genistein may be useful as medical chemical countermeasures against exposure to toxic chemical agents, such as phosgene, sulfur mustard gas, and phosphine gas. For example, a suspension formulation that contains genistein nanoparticles as the active ingredient may be used as a countermeasure to such exposure, increasing survival and decreasing pulmonary edema and other adverse effects associated with exposure to such agents. In some embodiments, the methods described herein can be used to mitigate acute and delayed respiratory effects in a chemically-compromised individual, and to prevent further lung damage. In some embodiments, the methods described herein also can be prophylactic, such that they can be used to protect an individual against the effects of exposure to a toxic chemical agent, preventing or reducing the effects should such exposure occur.

In one aspect, this document features a method for treating a subject to prevent or reduce one or more effects of exposure to a toxic chemical agent. The method can include identifying a subject as being at risk for exposure to the toxic chemical agent, and administering to the subject an effective dose of a composition comprising nanoparticulate genistein, wherein the composition has a nanoparticulate genistein concentration between about 250 mg/mL and about 500 mg/mL, and wherein the dose is effective to prevent or reduce one or more effects of exposure to the toxic chemical agent. The subject can be a human. The toxic chemical agent can be phosgene (e.g., phosgene gas or phosgene oxime), phosphine gas, or sulfur mustard gas. The subject can be likely to be present in an area of increased risk for exposure to the toxic chemical agent, or can be likely to be in an industrial setting associated with possible exposure to the toxic chemical agent. The genistein nanoparticulate composition can have a particle size distribution characterized by a d(0.5) less than or equal to 0.3 um. The composition can further include one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The nonionic surfactant can be present in an amount ranging from about 0.01% to about 10% by weight (w/w). The buffer can be a sodium phosphate buffer. The diluent can be a sodium chloride solution. The amount of water soluble polymer can be about 0.5% to about 15% (w/w). The composition can be in aerosol form and can include a propellant. The composition can be administered using a nebulizer. The nanoparticulate genistein can be present in the composition at an amount ranging up to about 50% (w/w) (e.g., about 20% to about 35% (w/w)). The composition can have a nanoparticulate genistein concentration of about 325 mg/mL. The composition can have a pH of about 2 to about 12. The method can include administering the composition to the subject orally, via intramuscular injection, via subcutaneous injection, via intravenous injection, or by inhalation. The method can include administering the composition to the subject within about 12 hours before potential exposure of the subject to the toxic chemical agent, or administering the composition to the subject within about 24 hours before potential exposure of the subject to the toxic chemical agent. The method can include administering the composition to the subject on a daily basis. The method can include administering the composition to the subject at a dose of about 0.5 g to about 2.5 g. The one or more effects of exposure to the toxic chemical agent can include one or more of chronic obstructive pulmonary disease, asthma, pulmonary fibrosis, lung lesions, bronchitis, emphysema, lipid peroxidation, and death. The one or more effects of exposure to the toxic chemical agent can include one or more of pulmonary edema, respiratory distress, oxidative stress, inflammatory cytokine responses, and airway inflammation.

In another aspect, this document features a method for mitigating one or more effects of exposure to a toxic chemical agent. The method can include identifying a subject as having been exposed to the toxic chemical agent, and administering to the subject a therapeutically effective dose of a composition comprising nanoparticulate genistein, wherein the composition has a nanoparticulate genistein concentration between about 250 mg/mL and about 500 mg/mL, and wherein the dose is effective to mitigate one or more effects of exposure to the toxic chemical agent. The subject can be a human. The toxic chemical agent can be phosgene (e.g., phosgene gas or phosgene oxime), phosphine gas, or sulfur mustard gas. The subject can have been exposed in an area of increased risk for exposure to the toxic chemical agent, or can have been exposed in an industrial setting associated with possible exposure to the toxic chemical agent. The genistein nanoparticulate composition can have a particle size distribution characterized by a d(0.5) less than or equal to 0.3 μm. The composition can further include one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The nonionic surfactant can be present in an amount ranging from about 0.01% to about 10% by weight (w/w). The buffer can be a sodium phosphate buffer. The diluent can be a sodium chloride solution. The amount of water soluble polymer can be about 0.5% to about 15% (w/w). The composition can be in aerosol form and can include a propellant. The composition can be administered using a nebulizer. The nanoparticulate genistein can be present in the composition at an amount ranging up to about 50% (w/w) (e.g., about 20% to about 35% (w/w)). The composition can have a nanoparticulate genistein concentration of about 325 mg/mL. The composition can have a pH of about 2 to about 12. The method can include administering the composition to the subject orally, via intramuscular injection, via subcutaneous injection, via intravenous injection, or by inhalation. The method can include administering the composition to the subject within about 60 minutes after exposure of the subject to the toxic chemical agent, or administering the composition to the subject within about 24 hours after exposure of the subject to the toxic chemical agent. The method can include administering the composition to the subject at least once a day for at least two weeks after exposure of the subject to the toxic chemical agent, or at least once a day for at least four weeks after exposure of the subject to the toxic chemical agent. The method can include administering the composition to the subject at a dose of about 0.5 g to about 2.5 g. The method can include administering the composition to the subject at a dose of about 1 g to about 2.5 g per day for about 7 days to about 28 days, and then administering the composition at a dose of about 0.2 g to about 1 g per day for about 4 weeks to about 16 weeks. The one or more effects of exposure to the toxic chemical agent can include one or more of chronic obstructive pulmonary disease, asthma, pulmonary fibrosis, lung lesions, bronchitis, emphysema, lipid peroxidation, and death. The one or more effects of exposure to the toxic chemical agent can include one or more of pulmonary edema, respiratory distress, oxidative stress, inflammatory cytokine responses, and airway inflammation.

In still another aspect, this document features the use of a composition containing nanoparticulate genistein for preventing or reducing, in a subject, one or more effects of exposure to a toxic chemical agent, wherein the composition has a nanoparticulate genistein concentration between about 250 mg/mL and about 500 mg/mL, and wherein the subject is identified as being at risk for exposure to the toxic chemical agent. The subject can be a human. The toxic chemical agent can be phosgene (e.g., phosgene gas or phosgene oxime), phosphine gas, or sulfur mustard gas. The genistein nanoparticulate composition can have a particle size distribution characterized by a d(0.5) less than or equal to 0.3 μm. The composition can further contain comprises one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The nonionic surfactant can be present in an amount ranging from about 0.01% to about 10% by weight (w/w). The buffer can be a sodium phosphate buffer. The diluent can be a sodium chloride solution. The amount of water soluble polymer can be about 0.5% to about 15% (w/w). The composition can be in aerosol form and can contain a propellant. The composition can be formulated for administration via a nebulizer. The nanoparticulate genistein can be present in the composition at an amount ranging up to about 50% (w/w) (e.g., at an amount of about 20% to about 35% (w/w)). The composition can have a nanoparticulate genistein concentration of about 325 mg/mL. The composition can have a pH of about 2 to about 12. The composition can be formulated for oral, intramuscular, subcutaneous, or intravenous administration, or for administration via inhalation. The composition can be for administration within about 12 hours before potential exposure of the subject to the toxic chemical agent, or for administration within about 24 hours before potential exposure of the subject to the toxic chemical agent. The composition can be for administration on a daily basis. The composition can be for administration at a dose of about 0.5 g to about 2.5 g. The one or more effects of exposure to the toxic chemical agent can include one or more of chronic obstructive pulmonary disease, asthma, pulmonary fibrosis, lung lesions, bronchitis, emphysema, lipid peroxidation, and death, or one or more of pulmonary edema, respiratory distress, oxidative stress, inflammatory cytokine responses, and airway inflammation.

In another aspect, this document features the use of a composition containing nanoparticulate genistein for mitigating, in a subject, one or more effects of exposure to a toxic chemical agent, wherein the subject is identified as having been exposed to the toxic chemical agent, and wherein the composition has a nanoparticulate genistein concentration between about 250 mg/mL and about 500 mg/mL. The subject can be a human. The toxic chemical agent can be phosgene (e.g., phosgene gas or phosgene oxime), phosphine gas, or sulfur mustard gas. The genistein nanoparticulate composition can have a particle size distribution characterized by a d(0.5) less than or equal to 0.3 μm. The composition can further contain comprises one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The nonionic surfactant can be present in an amount ranging from about 0.01% to about 10% by weight (w/w). The buffer can be a sodium phosphate buffer. The diluent can be a sodium chloride solution. The amount of water soluble polymer can be about 0.5% to about 15% (w/w). The composition can be in aerosol form and can contain a propellant. The composition can be formulated for administration via a nebulizer. The nanoparticulate genistein can be present in the composition at an amount ranging up to about 50% (w/w) (e.g., at an amount of about 20% to about 35% (w/w)). The composition can have a nanoparticulate genistein concentration of about 325 mg/mL. The composition can have a pH of about 2 to about 12. The composition can be formulated for oral, intramuscular, subcutaneous, or intravenous administration, or for administration via inhalation. The composition can be for administration within about 12 hours before potential exposure of the subject to the toxic chemical agent, or for administration within about 24 hours before potential exposure of the subject to the toxic chemical agent. The composition can be for administration on a daily basis. The composition can be for administration at a dose of about 0.5 g to about 2.5 g. The one or more effects of exposure to the toxic chemical agent can include one or more of chronic obstructive pulmonary disease, asthma, pulmonary fibrosis, lung lesions, bronchitis, emphysema, lipid peroxidation, and death, or one or more of pulmonary edema, respiratory distress, oxidative stress, inflammatory cytokine responses, and airway inflammation.

In another aspect, this document features an article of manufacture having an auto-injector containing a composition comprising nanoparticulate genistein, wherein the composition has a nanoparticulate genistein concentration between about 250 mg/mL and about 500 mg/mL. The genistein nanoparticulate composition can have a particle size distribution characterized by a d(0.5) less than or equal to 0.3 μm. The composition can further include one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The nonionic surfactant can be present in an amount ranging from about 0.01% to about 10% by weight (w/w). The buffer can be a sodium phosphate buffer. The diluent can be a sodium chloride solution. The amount of water soluble polymer can be about 0.5% to about 15% (w/w). The nanoparticulate genistein can be present in the composition at an amount ranging up to about 50% (w/w) (e.g., about 20% to about 35% (w/w)). The composition can have a nanoparticulate genistein concentration of about 325 mg/mL. The composition can have a pH of about 2 to about 12.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing the structures of phosgene, (left), phosgene oxime (center), and sulfur mustard (right).

FIG. 2 is a diagram showing the structure of genistein.

FIG. 3 is a diagram of an experimental protocol as described in Examples 6 and 7.

FIGS. 4A and 4B are graphs plotting survival of mice that were exposed to phosgene gas and then treated with vehicle or varying dosages of a genistein nanoparticle suspension (FIG. 4A), or with 400 mg/kg of the genistein nanoparticle suspension at varying times after phosgene exposure (FIG. 4B). *p<0.05 vs. untreated animals.

### DETAILED DESCRIPTION

Chemical agents have been used in conventional warfare against military personnel, but with the increased threat of terrorist activity, the focus in recent years has broadened to include possible civilian exposure. Acute and chronic pulmonary effects, as well as long-term eye and dermatological effects, are key etiologies that plague exposed individuals.

As used herein, the terms “toxic chemical agent” and “chemical agent” are meant to encompass gases, solids that release toxic vapors, and also the vapors released by such solids.

Inhalation of chemical agents such as phosgene, sulfur mustard gas, and phosphine gas primarily affects the respiratory system. Such chemical agents were initially used in World War I, and more recently were used in the Iran-Iraq war of the late 1980s, causing fatal and non-fatal causalities of greater than 1 million (Balali-Mood and Hefazi, Basic Clin. Pharmacol. Toxicol. 99(4):273-282, 2006; and Robinson, World Health Organization, 2004). Individuals exposed to sulfur mustard gas during the Iran-Iraq war still suffer from late toxic effects of this warfare (Ghanei and Harandi, Inhalation Toxicol. 23(7):363-371, 2011), including respiratory problems such as lung lesions (Khateri et al., J. Occ. Environ. Med. 45(11):1136-1143, 2003). No cure has been identified for these chemical agents, and patients are treated by supportive medical care such as steroids, ibuprofen, N-acetylcysteine, and positive airway pressure ventilation.

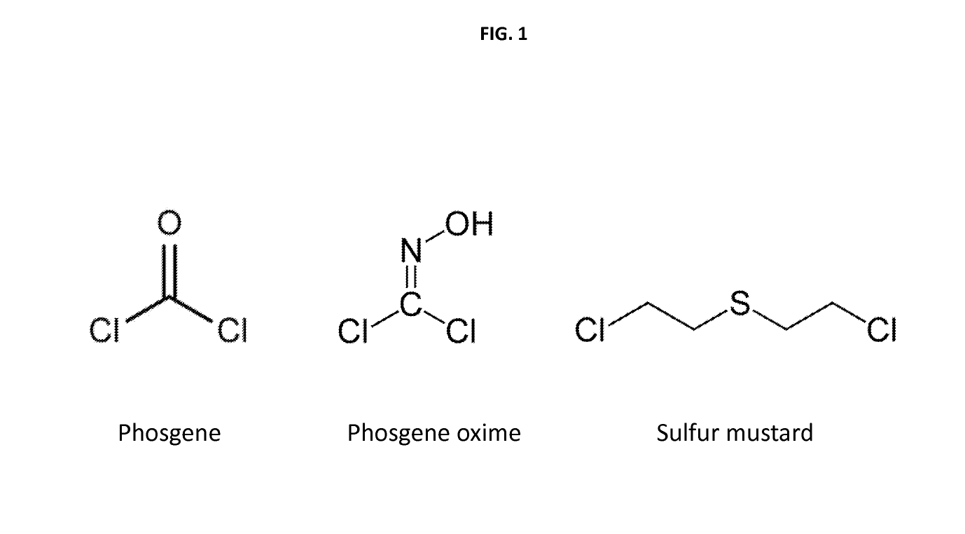

As used herein, the term “phosgene” is meant to encompass at least phosgene gas and phosgene oxime. Phosgene gas is a colorless gas with the formula COCl₂ (FIG. 1, left panel). Phosgene gas (CG) is highly toxic to the lung, inducing both acute and chronic effects that include upper and lower obstructive disease, airway inflammation, pulmonary fibrosis, chronic bronchitis, and acute respiratory distress (Borak and Diller, Envir 43(2):110-119, 2001). Exposure is associated with inflammatory and oxidative injury, resulting in respiratory tract damage and pulmonary symptoms. The mechanism of CG toxicity causes the release of sulfidopeptide leukotrienes and increases inflammatory cytokines. This leads to increased pulmonary capillary permeability and edema formation (Borak and Diller, supra; and Guo et al., J. Appl. Physiol. 69(5):1615-1622, 1990).

Phosgene oxime (CX) is an organic compound with the formula C12CNOH (FIG. 1, center panel). CX has a strong, disagreeable odor and a violently irritating vapor. The compound is a colorless solid, although impure samples often are yellowish liquids. CX is toxic by inhalation, ingestion, or skin contact, and the effects of exposure occur very rapidly. CX exposure can lead to respiratory tract effects such as irritation of the mucus membranes, runny nose, hoarseness, pulmonary edema, and pulmonary thrombosis. CX exposure also can lead to skin effects such as pain, itching, a hive-like rash, and blanching surrounded by a red ring that may appear almost immediately after exposure to CX; eye effects such as severe pain, itching, conjunctivitis, tearing, lid edema, blepharospasm, keratitis, iritis, corneal perforation, and temporary blindness; and gastrointestinal effects such as hemorrhagic inflammatory changes in the GI tract.

Phosphine gas (PH3) is a widely used fumigant and pesticide (Fluck, Inorganic Chemistry, Springer, pp. 1-64, 1973). PH3 poisoning is associated with a mortality rate greater than 70% (Murali et al., Clin. Toxicol. 47(1):35-38, 2009). Mechanisms of PH3 action may include disruption of energy metabolism, inhibition of respiration in mitochondria, and oxidative stress by generation of the reactive oxygen species (ROS) superoxide and hydrogen peroxide (Nath et al. Toxicol. 2011; 2011).

Sulfur mustard gas (SM) has the formula (Cl—CH₂CH₂)₂S (FIG. 1, right panel). SM is highly toxic to the lung, inducing both acute and chronic effects that include upper and lower obstructive disease, airway inflammation, pulmonary fibrosis, chronic bronchitis, and acute respiratory distress (Weinberger et al., Pulmonary Pharmacol. Ther 24(1):92-99, 2011; and Emad and Rezaian, CHEST J. 112(3):734-738, 1997). Exposure is associated with inflammatory and oxidative injury, resulting in respiratory tract damage and pulmonary symptoms. The mechanism of toxicity for SM involves DNA alkylation and consequent DNA damage, glutathione depletion, NF-κB pathway activation, and oxidative stress.

Other chemical agents that pose a threat via chemical warfare or industrial accident include, for example, acrolein, adamsite, allyl alcohol, ammonia solutions (>20%), anhydrous ammonia, arsenic trichloride, boron trichloride, boron trifluoride, boron trifluoride formulations with methyl ether, bromine, chlorine, chlorine dioxide, chloroacetone, chloroform, chloropicrin, chlorosulfonic acid, cyclohexylamine, diborane, dimethyl sulfate, epichlorohydrin, ethylenediamine, fluorine, formaldehyde, hexachlorocyclopentadiene, hydrazine, hydrochloric acid, hydrofluoric acid, hydrogen bromide, hydrogen chloride, hydrogen fluoride, hydrogen selenide, isopropylchloroformate, lewisite, methyl hydrazine, methyl isocyanate, nitric acid, nitric oxide, nitrogen mustard (HN-3), oleum, perchloromethylmercaptan, phosphorous trichloride, propyleneimine, sarin, anhydrous sulfur dioxide, sulfur tetrafluoride, sulfur trioxide, and titanium tetrachloride. The methods described herein also may be useful for preventing, reducing, or mitigating the effects of exposure to these chemicals.

Both immediate and long-term health effects are possible following exposure to chemical warfare agents. For example, acute lung damage typically becomes apparent at 30 ppm/min or greater for CG exposure. Pulmonary edema generally is present within about 12-16 hours after the insult, and death may occur about 24-30 hours after exposure (Robinson, supra; and Borak and Diller, supra). At very high concentrations (>200 ppm), death will occur within minutes. Severe acute SM exposure results in pulmonary edema and inflammation of the respiratory tract, with symptoms occurring within the first 24 hours, possibly followed by death. In addition to causing respiratory injuries, SM can be mutagenic, carcinogenic, and teratogenic

(Robinson, supra). Acute lung damage after PH3 exposure typically occurs within the first few hours, while pulmonary edema may be delayed for about 72 hours following exposure, depending on the concentration (Assem and Takamiya, Health Protection Agency, Institute of Environment and Health, Cranfield University, 2007). In addition, chronic bronchitis and emphysema can be long-term effects of PH3 exposure. These pulmonary insults reduce the functional capacity of the lung and, depending on their severity and extent, can be fatal.

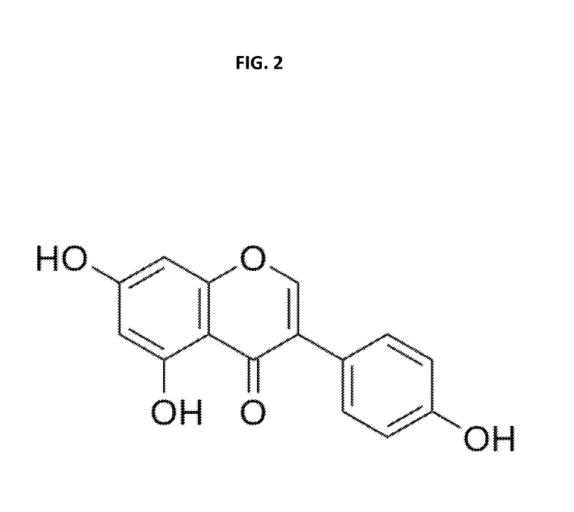

As described herein, compositions containing genistein can be used as medical countermeasures that can reduce or prevent chemical-induced pulmonary edema, and can increase survival after chemical exposure. Genistein (5,7-dihydroxy-3-(4-hydroxyphenyl)-chromen-4-one (IUPAC), 5,7-dihydroxy-3-(4-hydroxyphenyl)-4H-1-benzopyran-4-one, 5,7,4′-trihydroxyisoflavone, 4′,5,7-trihydroxyisoflavone) is a phytoestrogen in the category of isoflavones. Its chemical structure is shown in FIG. 2. Genistein is one of several known isoflavones that are normally found in plants. The main sources of natural genistein are soybeans and other legumes. Genistein also is commercially available, and may be obtained in synthetic, purified form (e.g., from DSM Nutritional Products, Inc., Parsippany, N.J.).

Genistein has strong antioxidant and anti-inflammatory properties (Verdrengh et al., Inflammation Res. 52(8):341-346, 2003; Polkowski and Mazurek, Acta Poloniae Pharmaceutica—Drug Research 57(2):135-155, 2000; and Kruk et al., Luminescence: J. Biol. Chem. Luminescence 20(2):81-89, 2005). Genistein also has effects on cell cycle division (Raffoul et al., BMC Cancer 6:107, 2006), and can inhibit protein tyrosine kinase activity, modulating signal transduction pathways involved in cell death and survival. The antioxidant properties of genistein may relate to genistein's ability to scavenge ROS, which are directly implicated in the formation of DNA double strand breaks. Thus, genistein may have beneficial effects in individuals with lung injury associated with exposure to toxic chemical agents. In addition to treating the underlying condition of chemical agent exposure, genistein may prevent further damage that causes pulmonary edema and other respiratory syndromes in the lung. Thus, genistein-containing compositions may be useful as a frontline therapy for treating individuals accidently or intentionally exposed to such toxic chemicals.

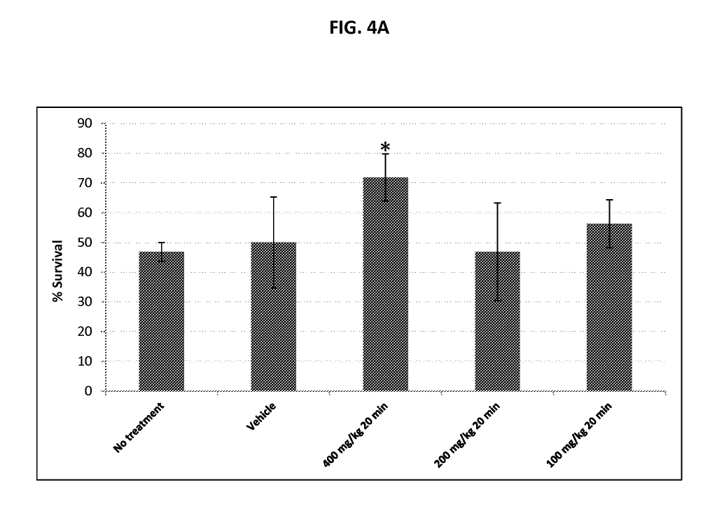

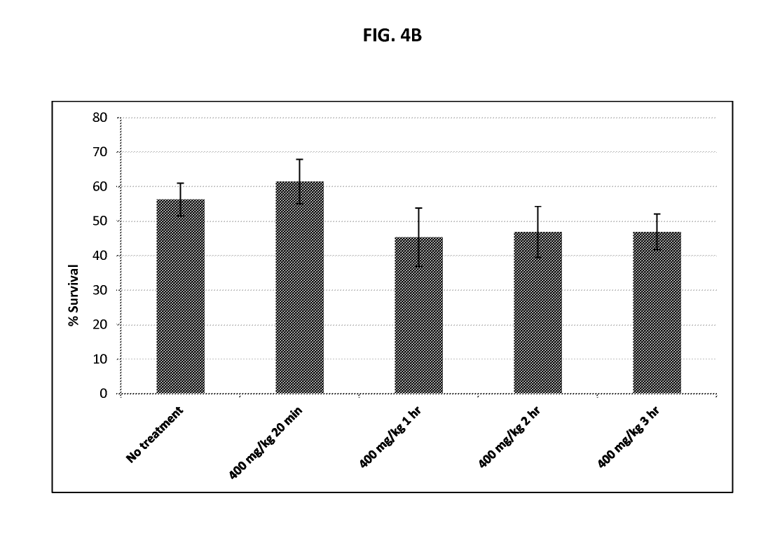

In some embodiments, a composition can include genistein nanoparticles, which can have improved oral and/or parenteral bioavailability as compared to genistein that is not in nanoparticle form. Nanoparticle formulations can contain sub-micron size genistein particles, which can be manufactured using a wet nanomilling process that reduces genistein to a median particle size of less than 0.2 μm. See, e.g., U.S. Patent No. 8,551,530, which is incorporated herein by reference in its entirety. Pharmacokinetic experiments using such a genistein nanosuspension in mice demonstrated dramatically increased oral bioavailability as compared to formulations containing non-micronized genistein. See, FIGS. 4-7 of U.S. Pat. No. 8,551,530.

In some embodiments, the genistein compositions used in the methods provided herein can be formulations that include genistein in a solution containing one or more pharmaceutically acceptable carriers, excipients, and/or diluents. In some embodiments, the genistein compositions used in the methods provided herein can be suspension formulations that include nanoparticulate genistein suspended in a medium containing one or more pharmaceutically acceptable carriers, excipients, and/or diluents. Pharmaceutically acceptable carriers, excipients, and diluents suitable for therapeutic use include those described, for example, in Remington's Pharmaceutical Sciences, Maack Publishing Co. (A. R. Gennaro (ed.), 1985). For example, in some cases, polyethyleneglycol (PEG) can be used as a carrier in a composition that also contains genistein that is not in nanoparticle form.

In some embodiments, a genistein composition can include a suspension containing nanoparticulate genistein suspended in an edible plant or animal oil (e.g., olive oil, sunflower oil, corn oil, soy oil, marine oil, coconut oil, palm oil, palm kernel oil, cotton seed oil, safflower oil, sesame oil, peanut oil, almond oil, cashew oil, pecan oil, pine nut oil, macadamia oil, orange oil, flax seed oil, lemon oil, walnut oil, borage oils, fish oils, and dairy derived fats).

In some embodiments, a genistein composition can include a suspension containing nanoparticulate genistein suspended in a medium including one or more water soluble polymers and one or more nonionic surfactants. See, e.g., U.S. Pat. No. 8,551,530.

Nonionic surfactants can facilitate wetting and aid in preventing agglomeration of the nanoparticulate genistein, for example. Suitable nonionic surfactants include, without limitation, polysorbates, poloxamers, polyoxyethylene castor oil derivatives, bile salts, lecithin, 12-hydroxystearic acid-polyethylene glycol copolymer, and the like. In some embodiments, a genistein composition can include a nonionic surfactant selected from the group consisting of polysorbate 80 (TWEEN® 80), polysorbate 20 (TWEEN® 20), Poloxamer 188, and combinations thereof. The total nonionic surfactant content in the genistein compositions provided herein can range from about 0.01% to about 10% by weight (w/w) (e.g., about 0.2% to about 5% (w/w), about 0.2% to about 2% (w/w), about 0.2% to about 1% (w/w), about 0.2% to about 0.6% (w/w), and about 0.2% to about 0.8% (w/w).

Water soluble polymers can serve to enhance the viscosity of a suspension and/or to stabilize nanoparticulate genistein against particle agglomeration or potential deleterious effects from other formulation components, for example. Water soluble polymers are pharmaceutically acceptable polymers that can be dissolved or dispersed in water. Suitable water soluble polymers include, without limitation, vegetable gums (e.g., alginates, pectin, guar gum, and xanthan gum), modified starches, polyvinylpyrrolidone (PVP), hypromellose (HPMC), methylcellulose, and other cellulose derivatives (e.g., sodium carboxymethylcellulose, hydroxypropylcellulose, and the like). In some embodiments, the genistein compositions described herein can include a poloxamer (e.g., Poloxamer 188) as a water soluble polymer. Poloxamer 188 is both a polymer and surfactant. The total water soluble polymer content in a genistein composition as provided herein can range from about 0.5% to about 15% (w/w) [e.g., about 1% to about 10% (w/w), about 10% to about 15% (w/w), about 12% to about 15% (w/w), about 1% to about 8% (w/w), and about 1% to about 5% (w/w)].

Carriers suitable for use in the genistein formulations described herein include pharmaceutically acceptable aqueous carriers such as, sterile water, physiologically buffered saline, Hank's solution, and Ringer's solution. The formulations also can contain one or more buffers [e.g., one or more citrate buffers, phosphate buffers, tris(hydroxymethyl)aminomethane (TRIS) buffers, and/or borate buffers], to achieve a desired pH and osmolality. Injectable pharmaceutical formulations typically have a pH in the range of about 2 to about 12. In some embodiments, the genistein formulations provided herein can have a pH that falls in a range that more closely approximates physiologic pH (e.g., about 4 to about 8, or about 5 to about 7).

The genistein compositions provided herein also can include one or more diluents. Suitable diluents include those selected from, without limitation, pharmaceutically acceptable buffers, solvents, and surfactants.

In some embodiments, a genistein composition can include PVP (e.g., 5% PVP-K17) and polysorbate 80 (e.g., 0.2% polysorbate 80), as well as phosphate buffered saline (PBS, e.g., 50 nM PBS). The composition also can include a diluent such as a sodium chloride solution. The particle size distribution of the genistein nanoparticulate composition can be d(0.5) <0.50 microns (e.g., d(0.5) <0.40 microns, d(0.5) <0.30 microns, or d(0.5) <0.20 microns). See, e.g., U.S. Pat. No. 8,551,530. It is to be noted that while such genistein formulations are characterized as suspensions, depending on the carriers, excipients, and diluents included in the suspension medium, a measurable amount of genistein also may be dissolved in the suspension medium.

Genistein exhibits low to virtually no solubility in several pharmaceutically acceptable solvents, but a nanoparticulate suspension of genistein as described herein can provide a high concentration of genistein. For example, a suspension of nanoparticulate genistein can incorporate genistein in amounts ranging from about 100 mg/mL to about 500 mg/mL (e.g., ranges from about 100 mg/mL to about 400 mg/mL, about 150 mg/ML to about 350 mg/mL, about 200 mg/mL to about 400 mg/mL, about 250 mg/mL to about 350 mg/mL, about 275 mg/mL to about 325 mg/mL, about 300 mg/mL to about 450 mg/mL, or about 350 mg/mL to about 500 mg/mL, or amounts of about 100 mg/mL, about 150 mg/mL, about 200 mg/mL, about 250 mg/mL, about 275 mg/mL, about 300 mg/mL, about 325 mg/mL, about 350 mg/mL, about 375 mg/mL, about 400 mg/mL, about 450 mg/mL, or about 500 mg/mL). The relative amount of genistein included in such a suspension can be varied to yield a formulation having a desired total content of genistein. For example, a suspension formulation as described herein can include up to about 50% (w/w) genistein [e.g., about 50% (w/w), about 45% (w/w), about 40% (w/w), about 35% (w/w), about 30% (w/w), about 25% (w/w), about 20% (w/w), about 15% (w/w), about 10% (w/w), about 40% to about 50% (w/w), about 35% to about 45%, about 30% to about 40% (w/w), about 25% to about 35% (w/w), about 20% to about 30% (w/w), about 20% to about 35% (w/w), about 15% to about 35%, about 10% to about 30%, or about 10% to about 25%]. In some embodiments, nanoparticle genistein suspensions can provide increased bioavailability of genistein as compared to the bioavailability of genistein provided by solution formulations (e.g., solutions containing a pharmaceutically acceptable PEG solvent or containing larger sized genistein material). As described in U.S. Patent No. 8,551,530, for example, the combination of high genistein loading and significantly increased bioavailability can provide advantages, such as facilitating administration of therapeutically effective amounts of genistein using much lower amounts of formulated drug substance, for example.

Genistein compositions can be formulated for administration by any suitable method, depending upon whether local or systemic treatment is desired and upon the area to be treated. For example, a genistein composition can be formulated for pulmonary administration (e.g., by inhalation or insufflation of powders or aerosols or a nebulized mist), oral administration, or parenteral administration (e.g., by subcutaneous, intrathecal, intraventricular, intramuscular, or intraperitoneal injection, or by intravenous drip), or by a combination of routes such as oral and parenteral administration. Administration can be rapid (e.g., by injection) or can occur over a period of time (e.g., by slow infusion or administration of slow release formulations, such as from subcutaneous drug depots, slow short term intravenous injections, or slow release oral formulations).

Compositions and formulations for parenteral administration include, for example, sterile solutions (e.g., sterile aqueous solutions or suspensions) that also can contain buffers, diluents, and/or other suitable additives (e.g., penetration enhancers, carrier compounds and other pharmaceutically acceptable carriers). Compositions formulated for parenteral delivery can be manufactured according to standard methods to provide sterile compositions deliverable via, for example, intravenous injection or infusion, intravascular injection, subcutaneous injection, or intramuscular injection. A genistein formulation (e.g., a suspension of nanoparticulate genistein) can be prepared to have a viscosity suitable for the desired route of parenteral administration, and can be manufactured and packaged in any manner suited to the desired application, including, without limitation, as a formulation deliverable via intravenous injection or infusion, intravascular injection, subcutaneous injection, or intramuscular injection. In some embodiments, a formulation as described herein can be contained in one or more pre-filled syringes or auto-injectors prepared for administration of a given dose or range of doses of genistein.

Genistein compositions also can be formulated for oral administration. Compositions and formulations for oral administration include, for example, powders or granules, suspensions or solutions in water or non-aqueous media (e.g., suspensions of genistein nanoparticles in edible oil), capsules, sachets, and tablets. In some embodiments, a genistein composition can be prepared as a liquid suspension that can be metered to deliver a desired dose, or can be incorporated into capsules (e.g., gelatin or soft capsules) suitable for delivery of liquid formulations. Alternatively, formulations for oral administration can be loaded into prefilled sachets or premetered dosing cups. In some embodiments, such genistein formulations also can include one or more pharmaceutically acceptable sweetening agents, preservatives, dyestuffs, flavorings, or any combination thereof.

In addition, genistein compositions can be formulated for pulmonary delivery, such as via an aerosol form. An aerosol typically is a system of solid particles or liquid droplets of a small size (generally less than 500 nm) suspended in air or another gaseous environment. Aerosols vary in size and composition, and can be naturally or synthetically generated. Aerosol formulations typically include a propellant, which is a chemical having a vapor pressure greater than atmospheric pressure at 40° C. Examples of propellants that can be used in pharmaceutical aerosols include, without limitation, chlorofluorocarbons, hydrocarbons, hydrochlorofluorocarbons, hydrofluorocarbons, and compressed gases. In some embodiments, aerosol formulations of genistein nanoparticles can be contained in one or more pre-filled inhalers prepared for administration of a given dose or range of doses of genistein.

In some embodiments, a genistein composition can be formulated for pulmonary administration in the form of a mist, such as via a nebulizer. A nebulizer is a device that typically uses oxygen, compressed air, or ultrasonic power to break up a solution or suspension into small aerosol droplets that can be directly inhaled from the mouthpiece of the device. A nebulizer can be powered mechanically (e.g., by a user's pumping action or actuation of a spring to increase and then quickly decrease the air pressure in a container holding the composition), in which cases a volatile liquid (e.g., alcohol) may be added to the composition to facilitate the increase in pressure. In some cases, a nebulizer can be powered electrically, using a vibrating mesh or a compressor, or an oscillator that generates a high frequency ultrasonic wave to cause mechanical vibration of a piezoelectric element.

Genistein compositions useful in the methods described herein can further include any pharmaceutically acceptable genistein salts, esters, or salts of such esters, or any other genistein compound which, upon administration to an animal such as a human, is capable of providing (directly or indirectly) biologically active genistein or an active metabolite or residue thereof. Accordingly, for example, provided herein are pharmaceutically acceptable salts of genistein, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of genistein (e.g., salts that retain the desired biological activity of genistein without imparting undesired toxicological effects). Examples of pharmaceutically acceptable salts may include, for example, salts formed with cations (e.g., sodium, potassium, calcium, or polyamines such as spermine), acid addition salts formed with inorganic acids (e.g., hydrochloric acid, hydrobromic acid, sulfuric acid, phosphoric acid, or nitric acid), and salts formed with organic acids (e.g., glucuronic acid, acetic acid, citric acid, oxalic acid, palmitic acid, or fumaric acid). Depending on the route of administration, for example, genistein may be sulfated or in glucuronic acid form.

Compositions also can include other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the genistein within the composition. The formulations can be sterilized if desired.

This document also provides methods for using genistein compositions as medical countermeasures against the effects of toxic chemicals such as phosgene, PH3, and SM gas. Such countermeasures can be used to prevent, reduce, or mitigate effects such as oxidative stress, pulmonary edema, and inflammatory cytokine responses, as well as lipid peroxidation, pulmonary fibrosis, and death.

In some embodiments, for example, this document provides methods for treating a subject to prevent or reduce one or more effects of exposure to a toxic chemical agent (e.g., phosgene, PH3, or SM gas). The methods can include identifying a subject as being at risk for exposure to the toxic chemical agent, and administering to the subject an effective dose of a genistein-containing composition. The subject can be, for example, a human or a non-human mammal Subjects at risk for exposure to a toxic chemical agent may be individuals present or likely to be present in areas of the world where chemical warfare may occur (e.g., military personnel or civilians living and/or working in areas prone to chemical warfare), or individuals present or likely to be present in an industrial setting in which exposure to toxic chemicals could occur. Such individuals can be treated with the genistein-containing composition prophylactically, before any chemical exposure has occurred. In some embodiments, such individuals can be treated on a daily or weekly basis (e.g., every day, about six days per week, about five days per week, about four days per week, about three days per week, or about two days per week), before potential exposure to a toxic chemical. In some embodiments, individuals can be treated within about 72 hours (e.g., within about 72 hours, about 60 hours, about 48 hours, about 36 hours, about 24 hours, about 18 hours, about 12 hours, about 10 hours, about 8 hours, about 6 hours, or about 4 hours) before potential exposure, such as before entering an area of increased risk for chemical exposure, to prevent or reduce potential harmful effects, should such exposure occur.

In some embodiments, this document provides methods for treating a subject to mitigate or prevent one or more effects of exposure to a toxic chemical agent (e.g., phosgene, PH3, or SM gas). The methods can include identifying a subject as having been exposed to the toxic chemical agent, and administering to the subject an effective dose of a genistein-containing composition. In some embodiments, the subject can be a human or a non-human mammal, and can be an individual present in an area of the world where chemical warfare has occurred, or an individual exposed to a toxic chemical in an industrial setting. Such subjects can be treated on an hourly, daily, or weekly basis after exposure, in order to mitigate harmful effects of exposure to the toxic chemical. In some embodiments, the genistein composition can be administered within about four weeks or less (e.g., within about three weeks, two weeks, or one week), or within about 96 hours or less (e.g., within about 72 hours, about 48 hours, about 24 hours, about 20 hours, about 18 hours, about 16 hours, about 12 hours, about 8 hours, about 6 hours, about 4 hours, or about 2 hours), or within about 60 minutes or less (e.g., within about 45 minutes, about 30 minutes, about 15 minutes, about 10 minutes, or about 5 minutes) after exposure to the toxic chemical agent. Administration can continue on an hourly, daily, weekly, or monthly basis to mitigate the effects of chemical exposure. For example, a genistein-containing composition can be administered one or more times daily, every other day, biweekly, weekly, bimonthly, monthly, or less often, for any suitable length of time after exposure to the chemical has occurred (e.g., for about a week, about two weeks, about three weeks, about a month, about six weeks, about two months, about three months, about six months, about a year, or more than a year after exposure).

The methods provided herein include administering to a subject a composition that contains genistein in any formulation suitable to deliver an effective dose of genistein to the subject, where the dose is effective to prevent, reduce, or mitigate effects of the chemical to which the subject has been or may be exposed. As used herein, a dose administered prophylactically that is “effective to reduce” the effects of the chemical is a dose that is sufficient to decrease one or more effects of chemical exposure (should such exposure occur) by at least ten percent (e.g., at least ten percent, at least 25 percent, at least 50 percent, or at least 75 percent) as compared to the level of the effects in a corresponding subject to which the composition was not administered. In some embodiments, an effective prophylactic dose can prevent development of one or more effects of exposure to the chemical. As used herein, a dose administered therapeutically that is “effective to mitigate” the effects of the chemical is a dose that is sufficient to reduce the effects of chemical exposure by at least ten percent (e.g., at least ten percent, at least 25 percent, at least 50 percent, or at least 75 percent), as compared to a corresponding subject to which the composition was not administered. Effective doses (e.g., therapeutically or prophylactically effective doses) can be effective to prevent, reduce, or mitigate effects of chemical exposure that include, without limitation, pulmonary edema, respiratory distress, airway inflammation, chronic obstructive pulmonary disease, asthma, pulmonary fibrosis, lung lesions, bronchitis, emphysema, oxidative stress, inflammatory cytokine responses, lipid peroxidation, and death.

In some embodiments, a composition can contain genistein (e.g., nanoparticulate genistein or genistein that is not in nanoparticle form), at a concentration between about 100 mg/mL and about 500 mg/mL (e.g., about 100 mg/mL to about 400 mg/mL, about 150 mg/ML to about 350 mg/mL, about 200 mg/mL to about 400 mg/mL, about 250 mg/mL to about 350 mg/mL, about 275 mg/mL to about 325 mg/mL, about 300 mg/mL to about 450 mg/mL, or about 350 mg/mL to about 500 mg/mL). Compositions containing nanoparticulate genistein can have a particle size distribution characterized by a median diameter [d(0.5)] that is less than or equal to 0.5 μm (e.g., less than or equal to 0.4 μm, less than or equal to 0.3 μm, or less than or equal to 0.2 μm). The composition also can contain one or more other components, as described herein (e.g., one or more pharmaceutically acceptable excipients that form a suspension medium, such as a water soluble polymer, a nonionic surfactant, a diluent, or a buffer).

The administering step can be accomplished via any suitable route. In some embodiments, for example, a genistein composition in aerosol form can be administered via an inhaler to achieve delivery to the lungs. In some embodiments, a genistein composition can be administered using a nebulizer to achieve delivery to the lungs. In some embodiments, a genistein composition containing a solution of genistein or a suspension of genistein nanoparticles can be administered orally or parenterally (e.g., by injection, such as subcutaneous, intravenous, or intramuscular injection).

The composition can be administered at a dose effective to prevent, reduce, or mitigate one or more effects of exposure to a toxic chemical. For example, a prophylactic or therapeutic dose can contain about 0.25 g to about 2.5 g of genistein (e.g., about 0.25 g., about 0.3 g, about 0.4 g, about 0.5 g, about 0.75 g, about 1 g, about 1.25 g, about 1.5 g, about 1.75 g, about 2 g, about 2.25 g, or about 2.5 g). In some embodiments, a genistein composition can be administered to a subject at a dose of about 5 mg/kg to about 500 mg/kg (e.g., about 5 mg/kg, about 10 mg/kg, about 25 mg/kg, about 50 mg/kg, about 75 mg/kg, about 100 mg/kg, about 200 mg/kg, about 300 mg/kg, about 400 mg/kg, or about 500 mg/kg).

In some embodiments, a therapeutic method can include administering a first dose of genistein for a first period of time after exposure to a toxic chemical, and then administering a second dose of genistein for a second period of time. The first dose can be higher than the second dose. For example, a method can include administering a genistein-containing composition at a dose of 0.25 g to about 2.5 g per day (e.g., about 0.25 g to about 2 g., about 0.5 g to about 2.5 g, about 1 g to about 2.5 g, about 1 g to about 2 g, or about 1.5 g to about 2.5 g per day) for about 7 to about 28 days (e.g., 7, 8, 9, 10, 11, 12, 13, 14, 15, 16, 17, 18, 19, 20, 21, 22, 23, 24, 25, 26, 27, or 28 days), and then administering the composition at a dose of about 0.1 g to about 1 g per day (e.g., about 0.1 g to about 0.7 g, 0.2 g to about 0.5 g, about 0.3 g to about 1 g, about 0.5 g to about 0.8 g, or about 0.5 g to about 1 g) for about 4 to about 16 weeks (e.g., about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, or about 16 weeks).

Genistein formulations (e.g., genistein solutions or nanoparticle suspensions) can be combined with packaging material and sold as kits for preventing, reducing, or mitigating the effects of chemical exposure. Thus, this document also provides articles of manufacture that can include one or more genistein-containing compositions. The articles of manufacture can further include, for example, buffers or other control reagents for reducing, preventing, or monitoring the effects of chemical exposure. Instructions describing how genistein formulations are effective for preventing, reducing, or mitigating damage from chemical exposure also can be included in such kits.

In some embodiments, an article of manufacture can include a genistein formulation (e.g., a suspension of nanoparticulate genistein) contained within a means for administration, such as an auto-injector or an inhaler. For example, an auto-injector can contain a suspension of nanoparticulate genistein at a concentration between about 250 mg/mL and about 500 mg/mL, where the genistein nanoparticulate composition has a particle size distribution characterized by a d(0.5) of 0.5 μm or less (e.g., 0.4 μm or less, 0.3 μm or less, or 0.2 μm or less). The genistein composition also can include other components (e.g., one or more pharmaceutically acceptable excipients), as described herein.

Components and methods for producing articles of manufacture are well known. In addition, in some embodiments, pre-made auto-injectors or inhalers can be obtained commercially, filled with a genistein nanoparticulate composition, and packaged as a kit for preventing, reducing, or mitigating the effects of chemical exposure.

The invention will be further described in the following examples, which do not limit the scope of the invention described in the claims.

### EXAMPLES

### Example 1

### Determining the Therapeutic Effect of a Genistein Nanoparticulate Composition after Exposure of Mice to Phosgene

Studies are conducted to evaluate the therapeutic potential of a genistein nanoparticulate composition as a medical chemical countermeasure. These studies are used, for example, to determine whether genistein treatment can restore the balance between chemical agent-induced oxidative stress signals and antioxidants while alleviating the chronic inflammation associated with exposure, to determine if dosing of a genistein nanoparticulate composition after exposure to phosgene increases 24 hour survival, to determine if dosing of the genistein nanoparticulate composition post exposure prevents and/or mitigates pulmonary edema and associated lung pathology, and to evaluate potential biomarkers (e.g., cytokines) in lung lavage fluid, by measuring oxidative stress-related protein levels, and through immunohistochemistry (IHC) of lung tissue. Outcome measures thus include 24 hour survival, pulmonary edema (wet/dry lung weights), levels of molecular markers (e.g., cytokines, LDH, total protein (albumin), and MPO) in BALF, and histopathology (H&E).

For these studies, male CD-1 mice are randomly assigned to study groups [e.g., control (ethanol- or air-exposed, vehicle treatment), genistein treatment, or agent (phosgene or vehicle)]. Four (n=4) trials are performed in the screening study, using a total of 192 mice. Animals are exposed to phosgene using established methods, and a single 200 mg/kg, 400 mg/kg, or 600 mg/kg dose of a genistein nanosuspension is given IM 15-60 minutes after exposure to the phosgene (Table 1). In further screening studies, genistein is administered prophylactically, prior to phosgene exposure (e.g., as a single dose given 15 minutes to 24 hours prior to phosgene exposure).

Survival is assessed 24 hours post phosgene exposure. Animals that survive to 24 hours are euthanized for analysis of wet/dry lung weights, and lavage fluid is collected for measurement of markers (e.g., cytokines, LDH, total protein, and MPO). Histopathology studies also are conducted. Samples from animals that fail to survive to 24 hours also are prepared for histopathology, should the animals be found soon after death. At the end of each experimental trial, lungs from the surviving animals are used as follows:

Trial 1: half for histopathology, half for BALF

Trial 2: half for wet/dry lung weights, half for BALF

Trial 3: half for histopathology, half for wet/dry lung weights

Trial 4: half for BALF, half for wet/dry lung weights

TABLE 1
Phosgene screening study

| | Number of mice | | |
|---|---|---|---|
| | Wet/dry lung | | |
| Screening Dose | weights | BALF | Histopathology |
| Vehicle | 12 | 12 | 12 |
| 200 mg/kg | 12 | 12 | 12 |
| genistein | | | |
| 400 mg/kg | 12 | 12 | 12 |
| genistein | | | |
| 600 mg/kg | 12 | 12 | 12 |
| genistein | | | |

A representative outline of an experimental protocol follows.

Animals: Adult male CD-1 mice (weight: 25-36 g) are obtained from Charles River (Wilmington, Mass.).

Screening phase: A single dose of genistein nanoparticulate composition is administered via IM injection, and also via oral gavage, at a dose of 200, 400, or 600 mg/kg/day. The genistein nanoparticulate composition is given at a time point from 15-60 minutes post chemical exposure, up to 24-72 hours post exposure. Alternatively, one or more prophylactic doses are given between 15 minutes and 24 hours prior to phosgene exposure.

Formulation: The genistein nanoparticulate composition contains 325 mg/mL genistein in 50 mM phosphate buffered saline (61 mM sodium chloride); 0.2% (w/v) polysorbate 80; 5% (w/v) polyvinylpyrrolidone K17.

Volume delivered per animal: For mice, about 0.005 mL/g weight (no more than 0.010 mL/g) is administered to each animal. All animals are dosed individually based on their weekly body weight.

Dilution: One mL of 325 mg/mL genistein nanoparticulate composition is diluted with vehicle solution (5.25 mL), for a total solution volume of 6.25 mL. The working oral suspension is dosed at 0.005 mL/g mouse body weight.

Vehicle: The control vehicle is the nanoparticulate composition minus the genistein.

Treatment planning: Treatment planning and chemical exposure are performed as previously described (Sciuto and Moran, J. Applied Toxicol. 21(1):33-39, 2001). Mice are placed into a 15.8 liter exposure chamber and simultaneously exposed whole-body to an amount of 32 mg/m³ (8 ppm) phosgene for 20 minutes, followed by a 5 minute room air washout.

Study design: Adult male CD-1 mice are randomized to the following groups (n=12/group): (1) Chemical agent+vehicle; (2) chemical agent+200 mg/kg genistein nanoparticulate composition; (3) chemical agent+400 mg/kg genistein nanoparticulate composition; and (4) chemical agent+600 mg/kg genistein nanoparticulate composition. Each group is repeated 4 times.

Physiologic assessments: The primary endpoint for the study is survival. Secondary endpoints include (1) lung edema, which is estimated based on left absolute lung wet weight; (2) oxidative stress, assessed using lung tissue lysates for measuring levels of thiobarbituric acid reactive substances (TBARS, a marker of lipid peroxidation) and glutathione (GSH, an antioxidant), as previously described (Ji et al., Inhalation Toxicol. 22(7):535-542, 2010); and (3) pathologic damage of lung tissue, which is assessed by harvesting and formalin fixing the right lung of each animal for histology based on hematoxylin and eosin staining In addition, lung tissue lysates may be subjected to western blot analysis to probe for signal transduction pathways involved in the therapeutic response. For example, NF-κB and Nrf2 pathways may be analyzed for alterations in protein expression. Growth factor (e.g., TGF-β) and matrix metalloproteinase (MMP) expression levels also may be assessed.

### Example 2

### Determining the Therapeutic Benefit of Genistein Nanoparticulate Composition on Delayed Effects Following Exposure of Mice to Phosgene

Delayed effects from phosgene exposure may present weeks to months post exposure. Survival from the acute effects of exposure increases the risk of chronic, long term effects, including death, pulmonary pneumonitis and fibrosis, kidney damage, gastrointestinal tract damage, brain damage, and/or cardiac damage. Using the methods described in Example 1, chronic effects on target tissue can be assessed histologically and biochemically in potential target organs at different times post exposure. Different repeat dose regimens also can be evaluated.

Thus, further studies are conducted to assess the therapeutic potential of the genistein nanoparticulate composition for preventing and/or mitigating delayed effects of phosgene exposure (e.g., survival and lung pathology), to correlate delayed pathological effects of phosgene exposure to changes observed in biomarkers, and to further evaluate the therapeutic potential of the genistein nanoparticulate composition by determining its effect on these biomarkers. The route (IM or oral) and dose of the genistein nanoparticulate composition is determined based on the results of the screening study described in Example 1. Outcome measures include 30 day and 60 day survival, pulmonary edema (wet/dry lung weights), levels of molecular markers in BALF (e.g., cytokines, LDH, total protein (albumin), and MPO), protein expression assessed using lung tissue lysates, and histopathology (H&E).

Animals are exposed to phosgene using established methods, and are treated pre-or post-exposure based on the studies described in Example 1. In particular, these studies involve a single or repeat dose of the genistein nanoparticulate composition, using a concentration of the genistein nanoparticulate composition determined in the studies of Example 1 (Table 2). Survival is assessed 30 days and 60 days post phosgene exposure. Fifteen animals that survive to 30 days are euthanized from each group. Of those, five are evaluated for wet/dry lung weights, five for biomarker levels in BALF, and five for histopathology. Animals that fail to survive to 30 days also are used for histopathology studies, should they be found soon after death. The remaining animals that survive to 60 days are euthanized, and a third are used for assessment of wet/dry lung weights, a third for biomarker levels in BALF, and a third for histopathology. Again, animals that survive to 30 days but fail to survive to 60 days are used for histopathology analysis, should they be found soon after death. In some cases, other organs (e.g., kidney, heart, and/or brain) also are assessed for histopathology.

TABLE 2
Chronic phosgene studies

| Experimental | | | |
|---|---|---|---|
| group | Phosgene exposure | Administration | Number of mice |
| Vehicle | Yes | IM or Oral | 60 |
| Vehicle | No | IM or Oral | 30 |
| Genistein | Yes | IM or Oral | 60 |
| (mg/kg TBD) | | | |
| Genistein | No | IM or Oral | 30 |
| (mg/kg TBD) | | | |

### Example 3

### Determining the Therapeutic Effect of a Genistein Nanoparticulate Composition after Exposure to Sulfur Mustard or Phosphine

Studies are conducted to determine the therapeutic potential of a genistein nanoparticulate composition as a medical chemical countermeasure, and to determine whether genistein treatment can restore the balance between chemical agent-induced oxidative stress signals and antioxidants while alleviating the chronic inflammation associated with exposure, to determine if dosing of a genistein nanoparticulate composition after exposure to SM or PH3 increases survival post exposure, to determine if dosing of the genistein nanoparticulate composition post exposure prevents and/or mitigates pulmonary edema and associated lung pathology, and to evaluate potential biomarkers (e.g., cytokines) in lung lavage fluid, by measuring oxidative stress-related protein levels, and through immunohistochemistry (IHC) of lung tissue.

Male Sprague-Dawley rats are used for the SM and PH3 exposure studies. Animals are randomly assigned to study groups [e.g., control (ethanol- or air-exposed, vehicle treatment), genistein treatment, or chemical agent (SM, PH3, or vehicle). Animals are subjected to inhalation of SM or PH3 using established methods. A single 200, 400, or 600 mg/kg intramuscular (IM) dose of a composition containing genistein nanoparticles is administered either immediately after exposure to the chemical, or 15 to 60 minutes after exposure. 24-hour, 48-hour, and 72-hour survival is determined Therapeutic effects of treatment on mitigating pulmonary edema, improving lung tissue histology, and modulating the oxidative stress response are assessed in the animals. Subsequently, the effectiveness of a multiple dosing scheme (e.g., every 12 hours, every 6 hours, etc.) on overall survival is assessed. The dose and treatment regimens with the greatest statistically significant increase in survival and decrease in pulmonary edema are identified.

A representative outline of an experimental protocol follows. Animals: Adult male Sprague Dawley rats (weight: 200-250 g) are obtained from Charles River (Wilmington, Mass.).

Screening phase: A single dose of genistein nanoparticulate composition is administered by IM injection (or alternatively by oral gavage) at a dose of 200, 400, or 600 mg/kg/day. The genistein nanoparticulate composition is given at a time point from 15-60 minutes post chemical exposure, up to 24-72 hours post exposure. Alternatively, one or more prophylactic doses are given between 15 minutes and 24 hours prior to exposure to the toxic chemical agent.

Formulation: The genistein nanoparticulate composition contains 325 mg/mL genistein in 50 mM phosphate buffered saline (61 mM sodium chloride); 0.2% (w/v) polysorbate 80; 5% (w/v) polyvinylpyrrolidone K17.

Volume delivered per animal: For rats, about 0.002 mL/g weight (no more than 0.005 mL/g) is administered to each animal. All animals are dosed individually based on their weekly body weight. Dilution: Five mL of 325 mg/mL genistein nanoparticulate composition is diluted with vehicle solution (13 mL), for a total solution volume of 18 mL. The working oral suspension is dosed at approximately 0.002 mL/g mouse body weight.

Vehicle: The control vehicle is the injectable nanoparticulate composition minus the genistein.

Treatment planning: Treatment planning and chemical exposure are performed as previously described (Anderson et al., Inhalation Toxicol. 8(3):285-297, 1996). For SM, rats are anesthetized with a combination of ketamine and xylazine. After induction of anesthesia, rats are intubated. SM diluted in absolute EtOH to the desired dose of 1.4 mg/kg is placed in a heated water jacketed glass vapor generator, and the rats are connected to this device and exposed for 50 minutes. Similar methods are used to expose rats to phosphine.

Study design: Adult male Sprague-Dawley rats are randomized to the following groups (n=12/group): (1) Chemical agent+vehicle; (2) chemical agent +200 mg/kg genistein nanoparticulate composition; (3) chemical agent+400 mg/kg genistein nanoparticulate composition; and (4) chemical agent+600 mg/kg genistein nanoparticulate composition. Each group is repeated 4 times.

Physiologic assessments: The primary endpoint for the study is survival. Secondary endpoints include (1) lung edema, which is estimated based on left absolute lung wet weight; (2) oxidative stress, assessed using lung tissue lysates for measuring levels of thiobarbituric acid reactive substances (TBARS, a marker of lipid peroxidation) and glutathione (GSH, an antioxidant), as previously described (Ji et al., Inhalation Toxicol. 22(7):535-542, 2010); and (3) pathologic damage of lung tissue, which is assessed by harvesting and formalin fixing the right lung of each animal for histology based on hematoxylin and eosin staining. In some cases, lung tissue lysates also are subjected to western blot analysis to probe for signal transduction pathways involved in the physiological response to both the chemical exposure and the corresponding therapeutic treatment.

### Example 4

### Determining the Therapeutic Benefit of Genistein Nanoparticulate Composition on Delayed Effects Following Exposure to a Toxic Chemical Gas

Delayed effects from SM or PH3 exposure may present weeks to months post exposure. Survival from the acute effects of exposure increases the risk of chronic, long term effects, including death, pulmonary pneumonitis and fibrosis, kidney damage, gastrointestinal tract damage, brain damage, and/or cardiac damage. Using the methods described in Example 1, chronic effects on target tissue can be assessed histologically and biochemically in potential target organs at different times post exposure. Different repeat dose regimens also can be evaluated.

Further studies are conducted to assess the therapeutic potential of the genistein nanoparticulate composition for preventing and/or mitigating delayed effects of SM or PH3 exposure (e.g., survival and lung pathology), to correlate delayed pathological effects of exposure to changes observed in biomarkers, and to further evaluate the therapeutic potential of the genistein nanoparticulate composition by determining its effect on these biomarkers. The dose of the genistein nanoparticulate composition is determined based on the results of the screening study described in Example 1. Outcome measures include 30 day and 60 day survival, pulmonary edema (wet/dry lung weights), levels of molecular markers in BALF (e.g., cytokines, LDH, total protein (albumin), and MPO), protein expression assessed using lung tissue lysates, and histopathology (H&E).

Animals are exposed to SM or PH3 using established methods, and are treated pre-or post-exposure based on the studies described in Example 1. In particular, these studies involve a single or repeat dose of the genistein nanoparticulate composition, using a concentration of the genistein nanoparticulate composition determined in the studies of Example 1. Survival is assessed 30 days and 60 days post exposure to the chemical agent. Animals that survive to 30 days are euthanized from each group. Of those, five are evaluated for wet/dry lung weights, five for biomarker levels in BALF, and five for histopathology. Animals that fail to survive to 30 days also are used for histopathology studies, should they be found soon after death. The remaining animals that survive to 60 days are euthanized, and a third are used for assessment of wet/dry lung weights, a third for biomarker levels in BALF, and a third for histopathology. Again, animals that survive to 30 days but fail to survive to 60 days are used for histopathology analysis, should they be found soon after death. In some cases, other organs (e.g., kidney, heart, and/or brain) also are assessed for histopathology.

### Example 5

### Developing GMP Grade Genistein Nanoparticles

GMP grade genistein nanoparticulate compositions are prepared, and the pharmacokinetics, toxicokinetics, and safety of IM administration are evaluated.

GMP manufacturing includes, for example, a scale up of the milling process used to produce the genistein nanoparticulate composition, bioanalytical testing for assay and related substances (concentration), analysis of nanoparticle size, and testing to assess the stability of the nanoparticles. Studies also are conducted to assess the systemic bioavailability (e.g., Cmax, Tmax, t½, AUC, elimination rate, and volume of distribution).

### Example 6

### Optimizing the Dose and Administration Route for Genistein Nanoparticulate Composition to Prevent/Mitigate Pulmonary Damage and Increase Survival after Phosgene Exposure

Based on the results of the experiments in Examples 1 and 2, studies are conducted to evaluate the efficacy of administering a genistein nanoparticulate composition orally vs. IM to animals exposed to phosgene. Outcome measures include 24 hour survival, pulmonary edema (wet/dry lung weights), levels of molecular markers in BALF (e.g., cytokines, LDH, total protein (albumin), and MPO), and histopathology (H&E).

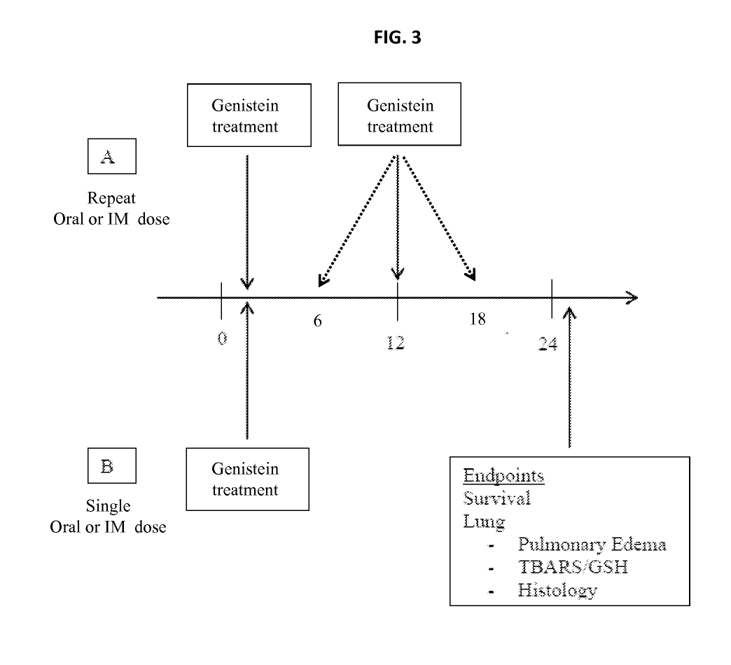

Subsequently, the effectiveness of a multiple dosing scheme (e.g., every 12 hours, every 6 hours, etc.) on overall survival is compared to a single dose (FIG. 3). The dose and treatment regimens with the greatest statistically significant increase in survival and decrease in pulmonary edema are identified. In addition to the dosing scheme, dose optimization is assessed. For example, utilizing the results of the screening studies, genistein nanoparticulate composition is administered via IM injection at a dose of 400 mg/kg/day or 200 mg/kg BID, starting 0.5 to 1 hour post chemical exposure, with a second dose given 6 or 12 hours post exposure. Alternatively, this dose optimization studies can be completed using a combination of both IM and oral delivery or a combination of both.

In these experiments, animals are exposed to phosgene using established methods, and the genistein nanoparticulate composition subsequently is administered at the dose determined to be most effective in the screening study described above. This dose is compared to an equivalent oral dose. Specifically, a single IM or oral dose of genistein nanoparticulate composition or vehicle control (Table 3) is given 15-60 minutes after exposure to phosgene, and survival is assessed 24 hours post phosgene exposure. Animals that survive to 24 hours are euthanized. Of those, a third are used for assessing wet/dry lung weights, another third for measuring marker levels in BALF, and the other third for histopathology. Histopathology samples also are prepared from animals that fail to survive to 24 hours, should they be found soon after death.

TABLE 3
Oral vs. IM studies after phosgene exposure

| Experimental group | Administration route | Number of mice |
|---|---|---|
| Vehicle | IM | 36 |
| Vehicle | Oral | 36 |
| Genistein (mg/kg TBD) | IM | 36 |
| Genistein (mg/kg TBD) | Oral | 36 |

### Example 7

### Optimizing the Dose and Administration Route for Genistein Nanoparticulate Composition to Prevent/Mitigate Pulmonary Damage and Increase Survival after SM or PH3 Eexposure

The effectiveness of a multiple dosing scheme (e.g., every 12 hours, every 6 hours, etc.) starting 0.5 to 1 hour post chemical exposure on overall survival is compared to a single dose (FIG. 3). The dose and treatment regimens with the greatest statistically significant increase in survival and decrease in pulmonary edema are identified. In addition to the dosing scheme, dose optimization is assessed. For example, utilizing the results of the screening studies, genistein nanoparticulate composition is administered via IM injection at a dose of 400 mg/kg/day or 200 mg/kg BID, starting 0.5 to 1 hour post chemical exposure, with a second dose given 6 or 12 hours post exposure. Alternatively, in some instances, these dose optimization studies can be completed using a combination of both IM and oral delivery.

In these experiments, animals are exposed to SM or PH3 using established methods, and the genistein nanoparticulate composition subsequently is administered at the dose determined to be most effective in the screening study described above. This dose is compared to an equivalent oral dose. Specifically, a single IM or oral dose of genistein nanoparticulate composition or vehicle control is given 15-60 minutes after chemical exposure, and survival is assessed 24, 48, and 72 hours post chemical exposure. Animals that survive to the end of the study are euthanized. Of those, a third are used for assessing wet/dry lung weights, another third for measuring marker levels in BALF, and the other third for histopathology. Histopathology samples also are prepared from animals that fail to survive to 24 hours, should they be found soon after death. These studies also provide an initial understanding of the molecular mechanism of genistein nanosuspension action by measuring biomarkers (e.g., cytokines) in lung lavage fluid, measuring oxidative stress-related protein levels, and conducting immunohistochemistry of lung tissue. This includes levels of molecular markers [e.g., cytokines, LDH, total protein (albumin), and myeloperoxidase (MPO)] in broncho-alveolar lavage fluid (BALF).

### Example 8

### Evaluating the use of a Genistein Nanoparticulate Composition to Prevent and/or Mitigate Systemic Toxicity and Increase Survival Following PH3 Gas Exposure

Studies are conducted to determine if dosing of a genistein nanoparticulate composition after exposure of rats to PH3 gas increases 12 hour survival, and to determine the optimal IM dose that improves 12 hour survival.

Animals are exposed to PH3 gas using established methods, and a single IM dose of vehicle control or genistein nanoparticulate composition at 200 mg/kg, 400 mg/kg, or 600 mg/kg (Table 4) is administered within 10 to 15 minutes of PH3 exposure. Survival is assessed 12 hours post PH3 exposure. Four (n=4) trials are performed in this screening study for each dose.

TABLE 4
PH₃ screening study

| Screening Dose | Number of rats |
|---|---|
| Vehicle | 12 |
| 200 mg/kg genistein | 12 |
| 400 mg/kg genistein | 12 |
| 600 mg/kg genistein | 12 |

Further experiments are conducted to evaluate the dosing regimen and effectiveness of the genistein nanoparticulate composition against systemic toxicity due to PH3 gas exposure. The IM dose of genistein nanoparticulate composition that provides a statistically significant increase in survival in the above-described screening study is used. These studies are used to compare the therapeutic potential of the genistein nanosuspension given as a single IM dose post PH3 gas exposure to q4 dosing for 12 hours (˜t=0, t=4, and t=8 hours), to determine if administration of the genistein nanoparticulate composition after PH3 exposure prevents and/or mitigates systemic toxicity, particularly in the liver, lung, brain, heart, kidney, and GI tract, and to further understand the mechanism of lung toxicity and begin to elucidate the role of the genistein nanoparticulate composition by measuring biomarkers [e.g., cytokines, LDH, total protein (albumin), and MPO] in lung lavage fluid. Outcome measures include 12 hour survival, levels of molecular markers in BALF, and histopathology (H&E) in organs/tissues such as lung, liver, brain, heart, kidney, and GI tract.

Animals are exposed to PH3 gas using established methods, and are treated with either a single IM dose of vehicle or genistein nanoparticulate composition, administered within 10 to 15 minutes after PH3 exposure, or with q4 dosing of vehicle or the genistein nanoparticulate composition for 12 hours (Table 5). Twelve hours after PH3 exposure, survival is assessed. Animals that survive to 12 hours are euthanized. Half of those are used for assessment of marker levels in BALF, and the other half for histopathology. Animals that fail to survive to 12 hours also are used for histopathology evaluation, should they be found soon after death.

TABLE 5
PH₃ dosing studies

| Screening Dose | Dosing regimen | Number of rats |
|---|---|---|
| Vehicle | Single IM | 18 |
| Genistein (mg/kg TBD) | Single IM | 18 |
| Vehicle | q4 for 12 hours | 18 |
| Genistein (mg/kg TBD) | q4 for 12 hours | 18 |

### Example 9

### Survival of Mice Exposed to Phosgene and Treated with Genistein Nanoparticle Suspension

Forty mice were pre-randomized to one of five treatment groups (untreated, vehicle, or 400, 200, or 100 mg/kg of genistein nanoparticle suspension), randomly assigned to a quadrant within the exposure chamber, and exposed whole body to ˜9-10 ppm phosgene gas for 20 minutes. About twenty minutes post-exposure, the animals received their respective treatment, administered intramuscularly. This was repeated three additional times, each time using five groups of eight mice each. Genistein nanoparticle suspension and vehicle were injected at 2.5 ml/kg.

Survival was assessed 24 hours after phosgene exposure. Of the three doses evaluated, the 400 mg/kg dose provided the best survival benefit (FIG. 4A). This improvement was significant (p=0.037) when compared to the untreated group, but not when compared to the vehicle treated group (p=0.06). Data were analyzed using a Fisher's exact test.

In subsequent experiments, forty mice were pre-randomized to one of five treatment times (untreated, 20 minutes, one hour, two hours, or three hours), randomly assigned to a quadrant within the exposure chamber, and exposed whole body to ˜9-10 ppm phosgene gas for 20 minutes. At their pre-assigned treatment time, all mice except those in the untreated group received an intramuscular dose of genistein nanoparticle suspension, given at 400 mg/kg. This was repeated three times, using five groups of eight mice each time.

Survival was assessed 24 hours after phosgene exposure. None of the four treatment group time delays provided a significant survival benefit when compared to the untreated group (FIG. 4B). Data were analyzed using a Fisher's exact test.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for preventing or reducing, in a human subject, one or more effects of exposure to a toxic chemical agent, comprising administering to the subject a composition comprising nanoparticulate genistein, wherein the composition has a nanoparticulate genistein concentration between about 250 mg/mL and about 500 mg/mL, and wherein the subject is identified as being at risk for exposure to the toxic chemical agent.

2. (canceled)

3. The method of claim 1, wherein the toxic chemical agent is phosgene gas.

4-7. (canceled)

8. The method of claim 1, wherein the genistein nanoparticulate composition has a particle size distribution characterized by a d(0.5) less than or equal to 0.3 μm.

9. The use of claim 1, wherein the composition further comprises one or more pharmaceutically acceptable excipients forming a suspension medium.

10-14. (canceled)

15. The method of claim 1, wherein the composition is in aerosol form and comprises a propellant.

16. The method of claim 1, comprising administering the composition via a nebulizer.

17-18. (canceled)

19. The method of claim 1, wherein the composition has a nanoparticulate genistein concentration of about 325 mg/mL.

20. (canceled)

21. The use method of claim 1, comprising administering the composition is via an oral, intramuscular, subcutaneous, or intravenous route, or via inhalation.

22. (canceled)

23. The use method of claim 1, comprising administering the composition is within about 24 hours before potential exposure to the toxic chemical agent.

24. (canceled)

25. The method of claim 1, comprising administering the composition is at a daily dose of about 0.5 g to about 2.5 g.

26. The method of claim 1, wherein the one or more effects of exposure to the toxic chemical agent comprise one or more of chronic obstructive pulmonary disease, asthma, pulmonary fibrosis, lung lesions, bronchitis, emphysema, lipid peroxidation, death, pulmonary edema, respiratory distress, oxidative stress, inflammatory cytokine responses, and airway inflammation.

27. (canceled)

28. A method for mitigating, in a human subject, one or more effects of exposure to a toxic chemical agent, comprising administering to the subject a composition comprising nanoparticulate genistein, wherein the subject is identified as having been exposed to the toxic chemical agent, and wherein the composition has a nanoparticulate genistein concentration between about 250 mg/mL and about 500 mg/mL.

29. (canceled)

30. The method of claim 28, wherein the toxic chemical agent is phosgene gas.

31-34. (canceled)

35. The method of claim 28, wherein the genistein nanoparticulate composition has a particle size distribution characterized by a d(0.5) less than or equal to 0.3 μm.

36. The use of claim 28, wherein the composition further comprises one or more pharmaceutically acceptable excipients forming a suspension medium.

37-41. (canceled)

42. The method of claim 28, wherein the composition is in aerosol form and comprises a propellant.

43. The method of claim 28, comprising administering the composition using a nebulizer.

44-45. (canceled)

46. The method of claim 28, wherein the composition has a nanoparticulate genistein concentration of about 325 mg/mL.

47. (canceled)

48. The method of claim 28, comprising administering the composition is for via an oral, intramuscular, subcutaneous, or intravenous route, or via inhalation.

49. The method of claim 28, comprising administering the composition is within about 60 minutes after exposure to the toxic chemical agent.

50-52. (canceled)

53. The method of claim 28, comprising administering the composition at a daily dose of about 0.5 g to about 2.5 g.

54. (canceled)

55. The method of claim 28, wherein the one or more effects of exposure to the toxic chemical agent comprise one or more of chronic obstructive pulmonary disease, asthma, pulmonary fibrosis, lung lesions, bronchitis, emphysema, lipid peroxidation, death pulmonary edema, respiratory distress, oxidative stress, inflammatory cytokine responses, and airway inflammation.

56-68. (canceled)
